(19) United States
(12) Patent Application Publication
(10) Pub. No.: US 2010/0158984 A1
(43) Pub. Date: Jun. 24, 2010

(54) Encapsulates

(75) Inventor: Fernando Qvyjt, Memphis, TN (US)

Correspondence Address: UNILEVER PATENT GROUP, 800 SYLVAN AVENUE, AG West S. Wing, ENGLEWOOD CLIFFS, NJ, 07632-3100, US

(73) Assignee: CONOPCO, INC., D/B/A UNILEVER, Englewood Cliffs, NJ (US)

(21) Appl. No.: 12/343,623

(22) Filed: Dec. 24, 2008

(51) Int. Cl. A61K 9/14; A61K 36/00

(52) U.S. Cl. 424/439; 424/489; 424/725

(57) ABSTRACT

An encapsulate comprises an outer shell and an inner core, the inner core comprising an ingredient selected from plant sterol compounds such as phytosterols and/or phytostanols and/or their esters, and at least one, preferably at least two, of the following groups: a, b, c, d, e, f, g, h, i, j, k.
a) Amino acids;
b) catechins;
c) rhubarb extract
d) quinine
e) pharmaceuticals, such as acetyl salicylic acid, salicylates, statins, DMSO, ranitidine
f) vitamins and minerals, especially cations such as potassium, calcium, magnesium, copper, iron, zinc, manganese, cobalt, molybdenum, chromium or combinations thereof)
g) an oil in water emulsion comprising 15-70 wt % of an oil having a saturated fatty acid content of at least 20 wt % and at least 10 wt % of the oil being solid at 37° C., 0.1-5 wt % protein emulsifier; and 25-85 wt % water.
h) A protein or proteinaceous material
i) A peptide or a polypeptide
j) Lycopene, Astaxanthine, Zeaxanthin and/or lutein
k) CLA

It can be expected that the presence of phytosterol and/or phytostanols and/or phytosterol and/or phytostanol esters will stabilize the other ingredient in the capsule providing, e.g. enhanced oxidative stability or controlled release by limiting diffusion.

### BACKGROUND OF THE INVENTION

In the last few years, weight reduction has been a focus of preventive medicine. Excessive weight is frequently cited almost daily in reports concerning type 2 diabetes. Moreover, obesity is often mentioned in discussions of other modern diseases, such as heart disease and even cancer.

Many consumers have turned to foods intended for weight management. Examples include nutrition bars, ready-to-drink beverages, powdered beverages and soups. Weight management foods can be used as meal replacements or for supplementing meals as a snack. One desired characteristic for weight management foods is the ability to leave the consumer satisfied following ingestion of a limited number of calories and to thereby delay the need for further intake of calories. That is, some weight management foods aim to increase satiety.

Some nutrients are reported to have enhanced effects on satiety. For example, proteins have been said to promote satiety better than other macronutrients. Other approaches to satiety have also been used. For example, US 2004/0258803 and US 2004/0258735 disclose use of encapsulated satiety agents wherein the satiety agent is released predominantly in the intestines, especially the ileum. Release of the satiety agent over more than one part of the intestine is said to be believed to aid efficacy of the agent.

Some ingredients themselves have been proposed for addition to foods for their satiety effects but cannot easily be added, for one reason or another, e.g., for reasons of taste or solubility.

Weight management is not the only health concern on the consumer's radar screen. Much public attention has been paid in recent years to a variety of food ingredients which reportedly have beneficial properties for the health. Among the most celebrated of these are the omega-3 fatty acids. One or more of these acids, and/or their sources, have been recommended for numerous conditions, such as high blood pressure, rheumatoid arthritis, undesirable cholesterol levels, mental acuity problems, infections, inflammation and elevated triglycerides. Two of the better known omega-3 fatty acids are DHA (docosahexaenoic acid) and EPA (eicosapentaenoic acid).

While it may be desirable to add omega-3 fatty acids and/or their sources to ingestable formulations, several characteristics of these nutrients make their inclusion in good tasting food products a challenge. For example, since these are polyunsaturated fatty acids, they have a tendency to oxidize and to produce an off-taste after a time.

Various references disclose food supplements which can be in the form of bars. These include WO 01/56402, Portman U.S. Pat. No. 6,051,236 (alpha lipoic acid which may be encapsulated in liposomes), Gilles et al. U.S. Pat. No. 6,248,375 (nutritional bar which may include fish oil), Keating et al. EP 768 043 (may include fish oil) and DeMichele et al. U.S. Pat. No. 6,444,700 (products which are useful for stress which may include fish oils; products in form of bars are discussed). WO 03/079818 discloses an alertness bar which may include sources of omega 3 fatty acids. Essential minerals are mentioned as well. Sears U.S. Pat. No. 6,140,304 (Eicotech Corp.) discloses a bar having a marine oil containing EPA. A ZONEPerfect® Nutrition Bar, Chocolate Mint flavor, available for sale in the United States at least as of Jul. 28, 2004, disclosed that it contains 3 mg of “OMEGA 3” and lists fish oil among its ingredients. The package is marked “BEST BY 04/05.”

Auriou US 2003/0165572 discloses water-dispersible powders including one or more phytosterols. Phytosterol micelles may contain significant quantities of other bioactives such as PUFA's carotenoids, eg lycopene, tocopherols and tocotrienols. A food or beverage comprising a phytosterol may also contin antioxidants such as ascorbate, amino acids such as arginine, vitamins and minerals. Among the many possible emulsifiers listed are alkyl betaine derivatives. Emulsion stabilizers may include amino acids proteins, peptides and lecithins. Excipients may include sugars.

Eppler et al. US Patent Application Publication No. 2005/0266137 discloses a food composition comprising from 5 to 30% hydrolyzed protein and a tryptophan source. An object is to provide meal replacement products or other calorie controlled products which have a high protein level but which also maintain good organoleptic properties. As food fat, fish oils, plant oils, seed oils, nut oils and others, or mixtures thereof, may be used. “Monosaturated” and/or polyunsaturated fats and mixtures thereof are said to be particularly preferred. Preferred polyunsaturated fats include omega 3 fatty acids. Optional ingredients include encapsulated satiety agents. These satiety agents may be encapsulated in any suitable cross-linked encapsulating agent whereby they are predominantly released in the intestines. Encapsulant materials comprising gelatin and at least one of gum arabic, carrageenan, agar agar, alginate or pectins, especially gelatin and gum arabic, have been found to be very suitable. The Eppler et al. compositions may also include one or more cholesterol lowering agents such as isoflavones, phytosterols, soy bean extracts, fish oil extracts, and tea leaf extracts. Aldred et al. US Published Patent Application No. US2005/0233045 also mentions encapsulated satiety agents, fish oils, sugar alcohols and phytosterols.

Yan U.S. Pat. No. 6,974,592 is directed to microcapsules including an agglomeration of primary microcapsules. Each individual primary microcapsule is said to have a primary shell which is encapsulated by an outer shell. The microcapsules may be prepared by providing an aqueous mixture of a loading substance and a shell substance, adjusting pH, temperature, concentration and/or mixing speed to form primary shells of shell material around the loading substance and cooling the aqueous mixture until the primary shells agglomerate, and an outer shell of shell material forms around the agglomeration. It is said that the loading substance may be virtually any substance which is not entirely soluble in the aqueous mixture. Preferably, the loading substance is a solid, a hydrophobic liquid or a mixture of a solid and a hydrophobic liquid. Omega-3 fatty acids and derivatives thereof and mixtures thereof are preferred. Examples of suitable derivatives are esters such as phytosterol esters. The microcapsules are said to be particularly suitable for biologically active substances for example, drugs, nutritional supplements, flavors or mixtures thereof. Claim 24 is directed to the invention of claim 2 wherein the loading substance for the microcapsule is a phytosterol.

Yan indicates that the shell can be any material that can form a microcapsule around the loading substance of interest. Examples include gelatins and polysaccharides. In example 1, gelatin and fish oil concentrate are included.

Magdassi et al. U.S. Pat. No. 6,303,149 is directed to a process for preparing 0.01-100 micron sol-gel microcapsules loaded with functional molecules and to products made by the process. The products can be used in various carriers, such as creams and lotions, processed food, sprays, paints, lacquers, coatings, plastics, detergents, etc. Examples including Beta-carotene and lycopene are given. It is said that the resulting powder can be easily suspended in hydrophilic phases such as water, milk, yoghurt, etc.

Ruseler-van Embden et al. US Patent Application Publication No. 2004/0166183 is directed to methods and means for preventing, treating or reducing inflammation comprising subjecting a mammal to a treatment with at least one inhibitor capable of inhibiting proteolytic activity. The composition can be in the form of a rinsing fluid, capsules, or diapers. A long list of potential ingredients is provided. The list includes acetamide, MEA, acetoglyceride, amino acids, beta-sitosterol, DHA, EPA, erythritol, and xylitol and mixtures thereof. Capsules which passage through the esophagus and stomach are mentioned.

Cooper et al. US Patent Application Publication No. 2003/0232796 is directed to nanoparticulate compositions comprising one or more polycosanols. In another aspect of the invention, novel combinations of polycosanols and other cholesterol lowering agents are described. Dosage forms may include capsules. The compositions may include one or more non-polycosanol active agents selected from the group consisting of cholesterol lowering agents, alkanoyl L-carnitines, antihypertensives, statins, sterols, and stanol. Cholesterol agents may include ACE inhibitors, nicotinic acid, niacin, bile acid sequestrants, fibrates, vitamins, and fatty acid derivatives such as fish oil. Other pharmaceutical excipients include xylitol as a sweetener and sorbitol and mannitol as diluents.

Ho et al. US Published Patent Application No. US 2006/0052438 is directed to certain compounds and their use, particularly to prevent cancer. Among the many other ingredients mentioned by Ho et al. are plant sterols, fish oil, sorbitol, mannitol, amino acids, vitamins, minerals, enzymes, conjugated linoleic acid (CLA), DHA, EPA,DMSO, alpha lipoic acid, green tea catechins, M-lycopene. Combinations of ingredients are mentioned. Ho et al.'s invention may be in capsule form. Nanoparticles and microparticles are mentioned.

Platt et al. US Published Patent Application No. US 2006/0052351 is directed to compositions comprising a combination of diacylglycerol and phytosterol and/or phytostanol ester(s) dissolved or dispersed in edible oil and/or edible fat, particularly olive, canola and/or fish oil in the manufacture of nutritional supplements and orally administrable pharmaceutical preparations. The supplements are said to be capable of reducing blood levels of both cholesterol and triglycerides and/or for lowering serum, serum LDL and macrophage oxidation levels. The supplements can be in the form of capsules.

Platt et al. US 2006/0233863 discloses esters of long chain polyunsaturated fatty acids with glycerol enriched with a mixture of esters of long chain polyunsaturated fatty acids with phytosterols and/or phytostanols, wherein the mixture of esters of long chain polyunsaturated fatty acids with glycerol are derived from an animal source such as fish oil, plant algae or microorganisms. In a preferred embodiment, a pharmaceutical dosage form is in the form of a capsule. In paragraph 191, Platt et al. indicate that since in the mixtures of the invention the phytosterol esters fraction also contain EPA and DHA fatty acids, the mixture of the invention can deliver RDI's of plant sterols by two user friendly capsules per day. By mixing long chain polyunsaturated fatty acid phytosterol esters with long chain polyunsaturated fatty acid-rich oil such as fish oil, a mixture with the recommended levels of omega-3 and phytosterols in a total amount of up to 2 g easily provided by 2 capsules.

Among processes disclosed for preparing capsules are various sol gel processes. Kong et al. WO 2006/084339 is directed to a process, which may involve a sol-gel process, for producing layered nanoparticles via a water-in-oil microemulsion. Hydrolyzable species such as TEOS may be used. The catalyst may be a strong acid, an organic acid, a base, an amine, a fluorde or a transition metal alkoxide. The inventors indicate that advanced controlled release technology may find applications not only in traditional applications for controlled release systems such as food, chemical, biocide, pesticide, pharmaceutical and cosmetic, but also in other areas.

Kong et al. WO 2006/133518 discloses a process for preparing particles having a hydrophobic material therein using sol-gel technology and multiple emulsions. A first emulsion is dispersed in a hydrophobic medium. The first emulsion comprises a hydrophobic phase dispersed in a hydrophilic phase. The hydrophobic phase comprises the hydrophobic material and the hydrophilic phase comprises a precursor capable of reacting to form a non-fluid matrix. The particles may be used to release a hydrophobic component which may be a drug or other therapeutic agent. The hydrophilic phase may be prepared by combining a crosslinkable species, such as TEOS, with water. High shear may be employed in forming the first emulsion. An acid or base catalyst may be used. On page 57, Kong et al. discuss the influence of synthesis pH on release rate. Kong et al. indicate that release of encapsulated oil can be controlled by controlling the porosity of the silica matrix, which may be controlled by adjusting the pH of the hydrophilic phase. At pH<2, the quantity of the pores and the size of the pores decreases, thus slowing the release of the oil.

Barbe et al. U.S. Pat. No. 7,258,874 and US 2005/0123611 disclose controlled release ceramic particles which may be prepared by a sol-gel process. Process 3 involves preparing a precursor solution including a gel precursor and an active ingredient, preparing a condensing solution by mixing a catalyst and a condensing agent which need not be water, combining the precursor solution and condensing solution to form an emulsion in the absence of surfactant, forming and aging controlled release ceramic particles. The catalyst may be an acidic or basic catalyst. The internal microstructure of the spheres can be precisely tailored by varying parameters such as water:alkoxide ratio, pH, alkoxide concentration, aging, and drying time and temperature. Hence the release ratio of the active ingredient is controlled by adapting the structure of the internal pore network to the properties of the active ingredient. Other patent documents cited by Barbe et al. as disclosing matrices prepared by sol-gel processes include U.S. Pat. No. 5,591,453, GB 1 590 574, WO 9745367 and WO 0050349.

Vanderhaeghe, L. R. and Bouic, P. J. D. The Immune System Cure: Optimize Your Immune System in 30 Days—The Natural Way! (New York City: Kensington Books, 1999), 48-50 provides the phytosterol content of selected foods.

### SUMMARY OF THE INVENTION

The invention is directed to the discovery that phytosterol compounds can have a stabilizing effect on other ingredients in an encapsulate. The invention further concerns encapsulates suitable for use in foods, especially foods designed for weight management. The invention is also directed to the foods and processes for making and using them. In particular, the invention is directed to encapsulates of certain ingredients, which may be health benefit agents, in combination with phytosterols and/or their esters wherein the phytosterols esters stabilize the other ingredients.

In accordance with one aspect of the invention, the encapsulate comprises an outer shell and an inner core, the inner core comprising an ingredient selected from plant sterol compounds such as phytosterols and/or phytostanols and/or their esters, and at least one, preferably at least two, of the following groups: a, b, c, d, e, f, g, h, i, j, k.
a) Amino acids;
b) catechins;
c) rhubarb extract
d) quinine
e) pharmaceuticals, such as acetyl salicylic acid, salicylates, statins, DMSO, ranitidine
f) vitamins and minerals, especially cations such as potassium, lithium, calcium, magnesium, copper, iron, zinc, manganese, cobalt, molybdenum, chromium, selenium, or combinations thereof)
g) an oil in water emulsion comprising 15-70wt % of an oil having a saturated fatty acid content of at least 20 wt % and at least 10 wt % of the oil being solid at 37° C., 0.1-5 wt % protein emulsifier; and 25-85 wt % water.
h) A protein or proteinaceous material
i) A peptide or a polypeptide
j) Lycopene, Astaxanthine, Zeaxanthin, betaine (trimethylglycine) and/or lutein
k) CLA

It can be expected that the presence of phytosterol and/or phytostanols and/or phytosterol and or phytostanol esters will stabilize the other ingredient in the capsule providing, e.g. enhanced oxidative stability or controlled release by limiting diffusion.

The weight ratio in the capsules of phytosterol and/or phytostanol and/or their ester(s) to ingredient the one or more of ingredients a-k may range from 100:1 to 1:100, preferably 90:1 to 1:90, more preferably 70:30 to 30:70, especially 60:40 to 40:60 up to 50:50.

Another advantageous combination of ingredients within capsules includes phytosterols and/or their esters with omega-3 oils or fatty acids and/or other polyunsaturated fatty acids.

The outer shell of the encapsulate may be a traditional food encapsulate such as gelatin, especially cross linked gelatin, but is more preferably made in a sol-gel process from metal or semi-metal alkoxide monomers or a partially hydrolyzed and partially condensed polymer thereof, or any mixture thereof.

In accordance with the invention, one of the ingredients to be encapsulated will be at least one phytosterol and/or phytostanol and/or their esters, especially their fatty acid esters. The sterols and stanols used in the present invention are those which are available from plants. Sterols can be classified in three groups, 4-desmethylsterols, 4-monomethylsterols, and 4,4′-dimethylsterols. In oils they mainly exist as free sterols and sterol esters of fatty acids although sterol glucosides and acylated sterol glycosides are also present. There are three major phytosterols, namely, beta-sitsterol, stigmasterol and campesterol. The phytostanols are the respective 5 alpha-saturated derivatives of phytosterols such as sitostanol, campestanol and their derivatives. Synthetic analogues of any of the phytsterols or phytostanols (which include chemically modified natural components) may also be used. Esters of acids other than long chain fatty acids, such as oryzanol, may be used. Preferably the phytosterol and/or phytostanol and/or ester is present at least 450 mg per 100 grams of the shell contents (exclusive of the shell itself). “Plant sterol compounds” as used herein shall, except as otherwise required by context, comprise, sterols found in plants including, without limitation, phytosterols, phytostanols, their esters, glucosides and acylated compounds.

Preferably, the encapsulate comprises at least two of the above components a through k plus omega 3 moieties and thereby contains at least 0.1 wt % omega-3 fatty acid moieties such as DHA, and/or an oil in water emulsion comprising 15-70wt % of an oil having a saturated fatty acid content of at least 20 wt % and at least 10 wt % of the oil being solid at 37° C., 0.1-5 wt % protein emulsifier; and 25-85 wt % water.

When an oil comprising at least 0.1 wt % of polyunsaturated fatty acid moieties is used, preferably at least 1 grams up to 8 grams per gram serving is used to cause a satiety effect. Preferably 3 grams of satiety agent reaches the ileum to achieve a satiety effect.

In a preferred embodiment, the invention combines at least two of the aforementioned components a through k, and preferably at least three, more preferably at least four, of the components, to deliver agents which have been reported to improve health. In addition, other health benefit agents such as omega-3 oils may be included in the capsules. The encapsulate may be designed to deliver the health benefit agent(s) and/or the satiety agent(s) to the small intestine of the person ingesting it, particularly to the ileum. While not wanting to be bound by theory, it is believed that delivery of the health benefit and/or other satiety agent(s) to the small intestine promotes satiety. Moreover, when including the health benefit agent in the encapsulate the health benefit agent serves the dual function of health benefit agent and promoting satiety at the same time.

The invention is further directed to food products, particularly weight management products, which include the encapsulates according to the invention. These include nutrition bars, ready-to-drink beverages, soups, sweet powders such as powdered beverages, baked goods, pasta, frozen confections and cereals. The invention is particularly directed to encapsulates, and food products containing the encapsulates, made using sol-gel technology.

The formulations according to the invention can be expected to have a very good shelf life, yet include one or more of groups a-k above and may include polyunsaturated fatty acids, especially fish oils, which generally have a tendency to oxidize. It is expected that the encapsulated oils will be less susceptible to oxidation and the off tastes which accompany oxidation.

If desired, any pro oxidants in the formulation such as copper and other metals, can are encapsulated, e.g., with carnauba wax and/or other waxes or with other encapsulating materials described herein.

The present invention is also directed to a process for inducing satiety in an animal such as a human by feeding the encapsulates prepared according to the invention to that individual. The invention is also directed to a process of preparing a food product by incorporating the encapsulates into the food product.

The advantages of the present invention can be at least two-fold. First, ingredients which would be difficult to include as a food ingredient for one reason or another, e.g. high oxidation rate, bitter taste, etc., can be encapsulatedwith phytosterols/esters or protect the ingredient and possibly to mask that property. Secondly, particularly when sol-gel encapsulation technology is used, the encapsulates can be prepared to disintegrate at high pH. pH is low in the stomach and higher in the small intestines, so such encapsulates will survive the high acid environment in the stomach and disintegrate in the intestines. This can be expected to increase the satiety effect, as explained in US 2004/0258803 and US 2004/0258735. In addition, the encapsulates can be used in foods such as beverages to visibly suspend particles.

Encapsulates of the invention are particularly suitable to use in weight management, e.g., for use to stimulate the so-called ileal brake wherein satiety agents are delivered directly to the ileum. Encapsulates of the invention can be tailored for use in ileal brake by synthesizing the encapsulates under conditions which promote resistance to degradation under the low pH/high acid conditions of the stomach and which promote release in the higher pH conditions of the intestines. Alternatively, or in addition, the encapsulates of the invention may be used to mask bitter, “fishy” or other undesirable taste properties.

Preferred processes for making the encapsulates of the invention involve Sol-gel technology. By use of acid or base catalysis, the form of the encapsulate and the reaction time can be influenced. Low pH catalysis favors formation of films, fibers, nanotubes and larger particles. The reaction is instantaneous. On the other hand, high pH catalysts favor spherulites and nanoparticles. Curing times for these of greater than 2 weeks are not uncommon.

Solgels can be formed at low temperatures and converted to glasses without a high temperature melting process (i.e. can be produced at room temperatures). The sol-gel process involves the evolution of inorganic networks through the formation of a colloidal suspension (sol) and gelation of the sol to form a network in a continuous liquid phase (gel). The precursors for synthesizing these colloids are a metal or metalloid element surrounded by various reactive ligands. Metal alkoxides, especially metal alkoxysilanes, are most popular because they react readily with water, for food purposes. Metal or semi-metal alkoxide monomers or a partially hydrolyzed and partially condensed polymer thereof, or any mixture thereof may be used as precursors.

The encapsulates of the invention are preferably made utilizing sol-gel technology by mixing, at room temperature and with high shear, tetraethoxy silane (TEOS) or another metal- or semi-metal alkoxide (or mixture thereof) with the core material which is to be encapsulated, e.g, phytosterol and one or more of ingredients a-k listed above, optionally with a source of omega-3 or other unsaturated fatty acids such as fish oil. Preferably the ingredients are mixed essentially without any solvent added, especially preferably without volatile solvent, e.g., hexane, cyclohexane, methanol or ethanol. Some solvent, such as alcohol, may be generated in the reaction, depending on the starting materials. It is not necessary or recommended to apply heat to the reaction, although if high melting ingredients (e.g. not liquid at room temperature) are used it may be desirable to heat them to melt them so that they are liquid when the reaction begins. It is generally preferred to conduct the process at ambient temperature, e.g., from 70-80° C., especially from 72-78° C. Typically, the reaction will be exothermic and the reaction temperature will increase as described below without application of heat. Preferably, the ingredients are mixed at a high shear prior to the addition of any catalyst, which is typically an acid or a base catalyst. Appropriate shear would preferably be within the range of from 600 to 15,000 rpm. The total time for imposing high shear preferably ranges to from 0.5 to 1440 minutes, preferably from 5 to 10 minutes. Shear may be continued at a lower rate after addition of the catalyst. Preferably, no heat or cooling is applied prior to the neutralization step of adding acid or base.

Any mixer capable of achieving high shear may be used, such as Silverson mixers. Unlike some processes in the art, in the preferred sol gel process the mixture of TEOS (or other metal or semi-metal alkoxide) is not dispersed to form an emulsion prior to addition of catalyst, nor is it usually preferred to include an HLB emulsifier. Indeed, preferably no emulsion is formed prior to addition of catalyst.

Generally, it is not necessary to form more than one layer around the encapsulate. That is, preferably the encapsulate is not multi-layer.

In a preferred process, the amount of water present prior to addition of acid or base catalyst during formation of the encapsulate is minimized. That is, the ingredients to be encapsulated and the materials used to form the shell are preferred to have no available water and are especially preferred to be water-free. Preferably, less than 1 wt % water form any source (even that normally present in some organic solvents like ethanol), more preferably, less than 0.5 wt % water, and most preferably less than 0.05 wt % water is present in the initial reaction mixture for forming the encapsulate.

The reaction mix used to form the encapsulated agents herein preferably have insubstantial amounts of organic solvent; more preferably they are free of organic solvent. Preferably, less than 1 wt %, especially less than 0.5 wt %, more preferably less than 0.05 wt % and most preferably no organic solvent is present. Solvents which are preferably present in insubstantial amounts, and which more preferably are excluded, from the preferred sol gel reaction used herein include organic solvents such as decanol, castor oil, hexane, acetone, THF, chloroform, dichloromethane, dimethylformamide, diethyl ether, tetrachloromethane, and alcohols such as ethanol, methanol, propanol, isopropanol, etc., as well as other organic solvents such as esters, ketones, aldehydes, nitriles and the like, paraffin oil, other hydrophobic solvents such as silicon oil, and combinations thereof.

Sol-gel is a technique for encapsulating ingredients which is flexible enough to accommodate different particle size, forms, application modes, etc. Bitter ingredients or others susceptible to oxidation or other degradation, or needed for slow release are among some ingredients which can be expected to benefit. The product can be made in various forms, such as monoliths, films, mono-sized powders and fibres.

The process for making the sol-gels according to the invention does not suffer from side reactions. Obviously, this would be a special concern for food applications. For instance, to be edible crystals should not be glassy; they should be amorphous and edible. Preferred products according to the invention are not glassy.

For a more complete understanding of the above and other features and advantages of the invention, reference should be made to the following description of the preferred embodiments.

### BRIEF DESCRIPTION OF THE DRAWINGS

FIG. 1 is a graph showing the results of the lipolysis experiment described in Example 4.

FIG. 2 is a graph showing the results of the experiment described in Example 10.

### DETAILED DESCRIPTION OF THE INVENTION

Amino acids which are preferably encapsulated are those having a bitter taste such as theanine, arginine, histidine, isoleucine, leucine, methionine, phenylalanine, tyrosine and valine. Theanine, for example, has been suggested for attention and mood improvement

Catechins are flavonoids and are polyphenolic antioxidants. They can be found in certain plants. They are present in the tea plant Camellia sinensis as well as the seeds of the cocoa plant, Theobroma cacao. The best known catechin is perhaps epigallocatechin gallate (EGCG). Other catechins include catechin (C), epicatechin (EC), gallocatechin (GC), epigallocatechin (EGC) and the gallates such as EGCG, gallocatechin gallate (GCG), Epicatechingallate (ECG), and catechin gallate (CG). Use of catechins such as epigallocatechin gallate have been suggested for reasons such as antioxidant and anti-inflammatory effects. In addition to tea and cocoa plants, catechins can be found in certain fruits, vegetables, and wine, e.g., bark (e.g. tea), grapes, wine, chocolate, apples, berries, etc.

Rhubarb extract has been reported to have beneficial health effects, including effects relating to weight management, hot flashes, and pancreatic cancer, among others.

Quinine is an alkaloid well known for its anti-malarial effects. Other benefits reported include fever reduction, analgesic effect and use as an anti-inflammatory.

Bitter tasting pharmaceutical compounds can benefit from encapsulation in accordance with the invention. Such pharmaceutical compounds include aspirin (acetyl salicylic acid), salicylates, ranitidine, urea, quinine, and acetaminophen.

Vitamins and/or minerals may be present in the encapsulates of the invention. Encapsulation in accordance with the present invention of vitamins can be expected to improve stability and reduce off-notes (e.g. for vitamins C, E, B12, etc.). Encapsulating compounds containing cations (e.g. potassium, calcium, magnesium, zinc, iron, manganese, cobalt, molybdenum, copper, chromium or combinations thereof) helps avoid undesirable reactions with other ingredients (e.g. alginates, PUFA's, MUFA's, vitamins, etc.) Encapsulated sources of copper or other pro-oxidants are especially preferably used in the foods of the invention. Encapsulated pro-oxidants are preferably present at a level of from 15 to 100% RDA.

In deciding whether vitamins and/or minerals are to be encapsulated with plant sterols in accordance with the invention, encapsulated in other capsules or present free of encapsulation, a method must be chosen so that processing and human absorption are not impaired.

Calcium may be present in the nutrition bars or other foods at from 0 to 100% of RDA, preferably from 10 to 30% RDA, especially about 25% RDA. The calcium source is preferably dicalcium phosphate. For example, wt. % levels of dicalcium phosphate may range from 0.5 to 1.5%. In a preferred embodiment, the product is fortified with one or more vitamins and/or minerals and/or fiber sources, in addition to the calcium source. These may include any or all of the following:

Ascorbic acid (Vitamin C), Tocopheryl Acetate (Vitamin E), Biotin (Vitamin H), Vitamin A Palmitate, Niacinamide (Vitamin B3), Potassium Iodide, d-Calcium Pantothenate (Vitamin B5), Cyanocobalamin (Vitamin B12), Riboflavin (Vitamin B2), Thiamine Mononitrate (Vitamin B1), Molybdenum, Chromium, Selenium, Calcium Carbonate, Calcium Lactate, Manganese (e.g., as Manganese Sulfate), Magnesium (e.g., as magnesium phosphate), Iron (e.g., as Ferric Orthophosphate) and Zinc (as Zinc Oxide).

The vitamins and minerals are preferably present at from 5 to 100% RDA, especially 5 to 50% RDA, most especially from about 15% RDA.

Vitamins and minerals may be present in encapsulates of the invention together with plant sterol compounds or in separate capsules or without encapsulation, if desired. Where it is preferred to encapsulate minerals and/or vitamins separately from the encapsulates of the invention, preferred are encapsulated copper salts such as microencapsulated cupric gluconate available from the Wright Group of Crowley, Louisiana. Another pro-oxidant copper salt which could benefit from encapsulation according to the present invention is copper sulfate. Encapsulated pro-oxidant salt products available from Wright include the following available under the name SuperCoat™: We 101266 (Iron), We 101265 (zinc): We 101270 (copper) and We 101267 (manganese). Encapsulated pro-oxidant salts are preferably present in the food of the invention at a level of from 0.3 to 0.85% by wt.

If desired, the pro-oxidants and other components of the invention may be encapsulated by coating with an edible wax, such as beeswax, carnauba wax, candellia wax, paraffin wax or mixtures thereof. Preferably the wax has a melting point greater than 65° C. Alternatively, the pro-oxidant can be coated with another coating material which provides resistance to food processing conditions/variables such as temperature, shear, moisture and oxygen levels, such as stearic acid, hard fats, edible waxes, cellulose and protein. Examples of hard fats include hydrogenated soy bean or cotton seed oils. Preferably, the pro-oxidants are completely coated by the wax or other encapsulating agent.

The vitamins and/or minerals may be included within, or external to, the encapsulates and any nuggets.

RDA as referred to herein is the Recommended Dietary Allowances 10^{th} ed., 1989, published by the National Academy of Science, National Academy Press, Washington, D.C.

Oil-in-water emulsions may be included in the shell to promote satiety. The oil in water emulsions used herein preferably comprise an oil in water emulsion including 15-70 wt % of an oil having a saturated fatty acid content of at least 20 wt %, at least 10 wt % of the oil being solid at 37° C., 0.1-5 wt % protein emulsifier; and 25-85 wt % water. Such emulsions may contribute to the satiety of the product without adversely affecting the taste, appearance or physical form of the foods (which may include beverages) into which it is incorporated. Protein emulsifiers are preferred for the emulsion. Especially preferred are egg or egg derived proteins, especially egg white-derived emulsifiers. Generally, the saturated fatty acid content is at least 25 wt % and preferably up to 90 wt %, especially from 25 wt % to 60 wt %, such as from 40 wt % to 60 wt %. However, the saturated fatty acid content of the emulsion can be as low as 0.5 wt % and above. Preferably the oil of the emulsion has a monounsaturated fatty acid content of at least 20 wt %, more preferably at least 30 wt %. The polyunsaturated fatty acid content of the emulsion is preferably at least 5 wt %.

The oil in water emulsions preferably comprise 0.3 to 3 wt % emulsifier. Preferred emulsifiers are selected from egg and egg-derived proteins. Egg white is very suitable. In an especially preferred embodiment, the emulsion comprises 20-60 wt % oil comprising fractionated palm oil, 0.5-3 wt % emulsifier comprising egg or egg derived protein and 40-60 wt % water. Where a blend of oils is included for the emulsion, it is preferred that the blend comprise fractionated palm oil, sunflower oil and soy bean oil.

The oil-in water emulsions may be prepared by any appropriate method. An especially suitable method is to melt the oil and to slowly add thereto the emulsifier under conditions of high shear while maintaining the oil in a molten state. This forms a dispersion of the emulsifier in the oil which is then slowly added to water which has been heated to at least the melting temperature of the oil, while mixing at high shear. Upon homogenization, lower homogenization pressures (e.g. 60-100 bar) result in a liquid emulsion and higher homogenization pressures (e.g. above 150) result in a thicker spoonable emulsion. Alternatively, the emulsion may be produced by first dissolving the emulsifier in the water, after which oil is added thereto. The oil-in-water emulsions are described in Bialek et al., published patent application US2006/0105093, the disclosure of which is herein incorporated by reference.

Lycopene is a carotenoid believed to have favorable health effects. Carotenoids, such as lycopene, have been investigated in connection with prostate cancer, macular degeneration, gingivitis, atherosclerosis, male infertility, oral submucous fibrosis, and oral leukoplakia. Preferred levels are 1-1000 mg per gram of sterols, preferably 2-30 mg of lycopene per 1 g of sterols. 2-30 mg per day is a preferred range of lycopene dosage. Other carotenoids which may be usefully employed in the present invention include astaxanthin, zeaxanthin and lutein. An advantage to inclusion of carotenoids, especially the aforementioned carotenoids, in a shell with a phytosterol or ester thereof is that the phytosterol limits the diffusion of the carotenoid when the shells are placed in water.

Conjugated linoleic acid (CLA) may be encapsulated herein. CLA has been mentioned as promoting favorable distribution of weight, e.g., more muscle mass and less fat. Sources of CLA include Loders Croklaan of Channahon, Ill.

In addition to the compounds mentioned, ingredients such as DHA, DHA/EPA and sources thereof, other omega-3 oils, polyunsaturated fatty acids (PUFA's) and sources thereof, omega 6, e.g., for hunger control, monounsaturated fatty acids (MUFA) and sources thereof such as olive oil and sunflower oil, other oils such as palm oil, other antioxidants, essential oils, sugar alcohols such as isomaltitol, sorbitol and/or erythritol, other flavonoids and other polyphenols, pro- and pre-biotics, flavors, aromas, glucosamine, inulin, Nutriose, sugars such as isomaltulose, dextrins such as cyclodextrin or other starches, glucose, mogrosides (Lo Han Kuo), and spices, resins or their extracts, are just a few of the potential active ingredients which may be encapsulated for use in foods in accordance with the invention in addition to the ingredients mentioned above.

End products including the encapsulates according to the invention may be prepared in various ways, e.g., extruded, baked, etc.

Encapsulation can be used in the present invention to promote slow release of aromas to influence taste.

Foods in which the encapsulates according to the invention can desirably be used include, without limitation, RTD (ready-to-drink) beverages, nutrition bars (including meal replacement and snack bars), powdered beverages (to be reconstituted by addition of liquids such as water or milk), bakery products and pasta. Preferably the foods of the invention include less than 2 wt % silica.

The omega-3 oil may comprise at least 0.1 wt % of DHA moieties, EPA moieties, ALA (alpha linolenic acid) moieties or mixtures thereof.

The shell can be any material that can form a microcapsule around the loading substance of interest, such as gelatins, polyphosphate, polysaccharides and mixtures thereof. Preferred polymer components include gelatin A, gelatin B, polyphosphate, gum Arabic, alginate, chitosan carrageenan, pectin, carboxymethyl cellulose (CMC), and mixtures thereof.

Where sol-gel is used to create a shell using tetraethoxy silane (TEOS), a preferred procedure is as follows. The basic reaction is TEOS+COREMATERIAL−(IN PRESENCE OF CATALYST)→(COREMATERIAL+SiO₂)+ETOH. Mix the core ingredients (eg, phytosterol and lycopene) and shell ingredient(s) (e.g., TEOS) at 10000 rpm for 5-10 minutes (high shear) in a Silverson mixer. Upon addition of the HCI catalyst, lower the shear to 4000 rpm. The catalyst is added when a homogeneous mixture is obtained. The reaction is initiated at room temperature, but is exothermic and typically reaches a temperature within the range of 120-180° F. or higher. Neutralization is initiated when temperature starts rising and chlorine is released. The gas is trapped in a small glass vessel (5 cc) containing 2 cc of water and causes a rapid drop in pH of water from 7 to 2 or below detected by using pH strips. The neutralization process is carried out very slowly at or above 140° F. with sodium hydroxide solution. The reaction is stopped at 160-190° F. After the reaction has been stopped, the product is mixed for 3 minutes at 200 rpm and then washed with aqueous ethanol, e.g., ethanol 95%, to remove NaOH. The product is then vacuum dried or dried using an alternative drying process. Sol gel clusters of up to 2 cm are typically obtained. The clusters are then ground gently.

Examples of metals and semi-metals, the alkoxides of which can be used in the encapsulating material, include silicon, zinc and zirconium.

Where the sol gel encapsulate is being prepared for the purpose of inducing satiety, or for some other reason an acid-resistant shell is desired, an acid catalyst is employed as described above, preferably hydrochloric acid. Prior to addition of the catalyst, the ingredients are mixed at a shear rate of from 600 to 5,000 rpm, especially about 4000 rpm for a period of from 5 to 300 seconds. The catalyst is then added. As mentioned, the reaction is an exothermic one, so the temperature of the reaction mixture increases as the reaction proceeds. At a temperature within the range of 90 to 158° F., especially 140° F., the mixture is neutralized slowly by gradual addition of a base, such as sodium hydroxide.

After neutralization, when the reaction reaches a temperature within the range of 135 to 203° F., especially 190° F., the reaction is stopped by reducing the shear because of the fast condensation and polymerization. Then the reaction mixture is mixed for from 5 to 300 seconds, especially 180 seconds at a shear rate of from 50 to 600, especially 200 rpm and subsequently washed with a solvent such as an alcohol, like 95% ethanol, to remove salt produced by the neutralization reaction. The mixture is then dried by vacuum drying or some other drying process.

Upon drying, sol gel clusters of up to 2.5 cm or so in length are produced. The clusters are then subjected to a gentle grinding step. The grinding step may, for instance, be effected using a mortar, balls or hammer grinders or dull blade grinders. The result is a sol gel encapsulation of the core material. The encapsulate (including the shell) preferably has a diameter of above 10 nanometers up to and including 200 microns. Most preferably, less than 2% by weight of the encapsulates have a diameter of 100 nanometers or less and less than 98 wt % has a diameter of above 200 microns. The resulting shell is resistant to, and remains intact upon, exposure to an acidic environment. The shell loses its integrity with higher pH's starting at about 7.2. These acid-resistant shells are expected to be particularly useful for applications where it is desired to deliver a core ingredient to the ileum of the digestive system to produce or enhance the sensation of satiety. The shells are resistant to very acidic environments, so it can be expected that they will retain their integrities in the stomach and stay intact until they pass into the small intestine. Experiments have shown that at the pH of the small intestine as the shells proceed through the intestines, some shells can be expected to lose their integrity over time, but a sufficient number would retain their integrity sufficiently to deliver the core product, e.g., lycopene and phytosterol, into the ileum.

Where acid catalysis is employed for sol-gel, it is preferred that the encapsulate is at least 5 wt % silica (silica 5 wt % of combination of shell and its contents) to avoid having the shell open prematurely in the stomach. If a minimum of around 5 wt % silica is used, it is believed that sufficient product will release in the ileum for a satiety effect. At less than about 5 wt % silica, the capsule may lose its integrity in the stomach, even with use of an acidic catalyst, such that the shell's content will be depleted in the stomach and the ileal brake effect may not materialize.

For encapsulates which are prepared for a reason other than enhanced satiety and other than having a shell which disintegrates upon exposure to an acidic environment, the process for preparing the encapsulate is the same, except that a base such as sodium hydroxide is used as the catalyst and an acid such as hydrochloric acid is used for neutralization. The resulting shell loses its integrity in an acidic environment. The resulting product can then be expected to be more water soluble and can dissolve in the stomach. An example would be for the encapsulation of an ingredient to mask bitterness or improve water solubility.

Preferably the silica shell prepared using sol-gel completely surrounds the shell contents.

Encapsulating can provide numerous possible benefits, such as stability from oxidation, masking bad taste, mouthfeel or aroma of an ingredient, improving or imparting water solubility, controlled release, reduced ingredient interaction. The encapsulate has the advantage of masking many undesirable tastes, in which case desirably the encapsulated ingredients may be combined with sweeteners such as sugars, artificial sweeteners such as high intensity artificial sweeteners, FOS, bitterness masking agents, etc. in case the consumer accidentally bites into and opens the capsule.

When sol gel is used, the reaction mix used to form the encapsulated agents herein preferably are free of surfactants, particularly HLB surfactants, and especially cationic surfactants. Preferably, less than 1 wt %, especially less than 0.5 wt %, more preferably less than 0.05 wt % and most preferably, no surfactant is present. Surfactants which are preferably excluded from the sol gel reaction herein include ammonium cations such as quaternary ammonium cations like cetyltrimethylammonium chloride, or tertiary ammonium cations, alkyl trimethyl ammonium chloride or bromide surfactants, hexadecyltrimethylammonium bromide, Polysorbate 20, Polysorbate 40, Polysorbate 60, Polysorbate 65, Polysorbate 80, Polysorbate 85, Sorbitan laurate, Sorbitan palmitate, Sorbitan stearate, Sorbitan tristearate, Sorbitan oleate, Sorbitan sesquioleate, trioleate Sorbitan, Simulsol 988/989 (PEG-7 Hydrogenated Castor oil), PEG-35-Castor oil, PEG-40 Castor Oil, PEG-25-Hydrogenated Castor oil, PEG-40-Hydrogenated Castor oil, PEG-60-Hydrogenated Castor oil, sorbitan esters, sodium oleate and poloxamers. Of course there may be situations where it is desirable to encapsulate an agent which has some emulsifying or other surface active properties, in which case such agent will be included in the reaction mix.

The sol gel reaction mix used to form the encapsulated agents herein preferably are free of viscosity modifying agents. Preferably, less than 1 wt %, especially less than 0.5 wt %, more preferably less than 0.05 wt % and most preferably no viscosity modifying agents are present. Viscosity modifying agents which are preferably excluded from the sol gel reaction herein includes without limitation, hydroxypropyl cellulose, ethyl cellulose, other celluloses such as, for example, hydroxypropyl methyl cellulose, methyl cellulose, hydroxyethyl cellulose and the like, acrylates such as, for example, sodium acrylate copolymers, paraffinium liquidum and PPG-1 trideceth-6, as well as PVP, maltodextrin, xantham gum, carbomers, lecithins, guar gum and wax.

The sol gel reaction mix used to form the encapsulated agents herein preferably are free of ingredients other than the core material to be encapsulated, the sol-gel precursor (e.g. metal or semi-metal alkoxide), the catalyst and ultimately the component (usually acid or base) needed to stop the reaction. Examples of such preferably excluded components include thickeners and gelling agents, anti-caking agents, anti-foaming agents, water soluble polymers, bulking agents, carriers and carriers solvents, emulsifying salts, firming agents, flavor enhancers, flavor treatment agents, foaming agents, glazing agents, humectants, modified starches, packaging gases, propellants, raising agents and sequestrants. Preferably less than 1 wt %, especially less than 0.5 wt %, more preferably less than 0.05 wt % and most preferably none of such additional components are present in the encapsulation reaction mix. Examples of preferably excluded water soluble polymers include uncharged water soluble polymers such as poly(ethylene oxides), poly(vinylpyrrolidones), poly(acrylamides), polyols such as poly(ethylene glycols), polyols with formamide, ionic polymers such as polyacrylates, poly(alkali metal styrene sulfonates), poly(allylamines) and combinations thereof.

In its preferred form, the present invention involves encapsulating the active ingredients, i.e., forming a shell around the ingredient rather than placing the ingredients in a pre-formed capsule. As indicated above, preferably a single encapsulation procedure is used; that is; it is not preferred in the present invention to encapsulate the encapsulations so as to obtain two or more shells.

Where the sol-gel technology is not employed to make the encapsulates, other methods may be used. For example, zein may be used as an encapsulating agent.

Other technologies which may be used to prepare encapsulates include those of the following, which are hereby incorporated by reference: Yan U.S. Pat. No. 6,974,592, Magdassi et al. U.S. Pat. No. 6,303,149, Lapidot et al. U.S. Pat. No. 6,238,650, Garti et al. US Patent Application Publication No. 2003/0228395, Garti et al. US Patent Application Publication No. 2003/0232095, Ruseler-van Embden et al. US Patent Application Publication No. 2004/0166183.

The invention is further directed to food products, particularly weight management products, which include the encapsulates according to the invention. Foods in which the encapsulates according to the invention can desirably be used include, without limitation, RTD (ready-to-drink) beverages, nutrition bars (including meal replacement and snack bars), sweet powders such as powdered beverages (to be reconstituted by addition of liquids such as water or milk), bakery products and pasta. Preferably the foods of the invention include less than 2 wt % silica. Other foods for which the invention is believed to be useful include soups, baked goods, frozen confections, such as ice cream, sorbet and frozen yogurt, and cereals.

Preferably, foods of the invention include the encapsulate at a level to provide at least 0.1 wt. % phytosterol or phytostanol moiety, preferably at least 1 wt. %, more preferably at least 5 wt. %, especially at least 8 wt. %. Preferably, foods of the invention include at least 0.5%, especially at least 2%, more preferably at least 4% by wt. of the encapsulate of the invention. For ileal brake, it is preferred that from 1-8 g, especially at least 3 g of the satiety agent reach the ileum.

An advantage of the present process is that the solgel process of the invention fully encapsulates active ingredients.

The particle sizes of our encapsulates can vary from nanoparticles, e.g., as small as 1×10⁻⁹ meters up to, preferably 200 microns, especially from 10 nanometers to 5 microns. More preferred are particle sizes above 100 nanometers, especially more than 2% of the average particle size is greater than 100 nm. Preferably less than 2% of the particles by weight are below 100 nm, more preferably less than 0.1% of the particle by weight are below 100 nm, most preferably less than 0.005 wt % and especially no particles are below 100 nm. The particles size of the encapsulate depends upon factors such as the process conditions, for example, time, temperature, shear, the catalyst employed, and the nature of the core.

The unsaturated fatty acids can be present as free fatty acids, but more typically will be present esterified to glycerol as mono-, di- or most preferably tri-acylglycerols. Unless otherwise required by context, references to any unsaturated fatty acids herein includes also reference to sources thereof such as triacylglycerols.

Preferably, the encapsulate comprises at least four of the above components a-k, preferably a sugar alcohol, sorbitol and/or erythritol, a sugar such as isomaltulose, a dextrin like cyclodextrin, a phytosterol, and an oil containing at least 0.1 wt % omega-3 fatty acid moieties such as DHA.

In place of or in addition to isomaltitol, various sugar alcohols may be used, such as maltitol, sorbitol and/or erythritol.

The omega-3 oil may comprise at least 0.1 wt % of DHA moieties, EPA moieties, ALA (alpha linolenic acid) moieties or mixtures thereof.

In addition to ingredients a-k above, the present invention may be used to incorporate any polyunsaturated fatty acid in the food and mixture thereof, and most especially to incorporate omega-3 and/or omega-6 fatty acids and/or esters of omega 3's and/or 6's and mixtures thereof. Among the polyunsaturated fatty acids for which the invention may be useful are included arachidonic acid, docosahexaenoic acid (DHA), eicosapentaenoic acid (EPA), linoleic acid, linolenic acid (alpha linolenic acid) and gamma-linolenic acid and mixtures thereof. The fatty acids and/or sources may be included in combinations described above or may be encapsulated by themselves or with other components.

Among sources for the unsaturated acids which are encapsulated in accordance with the invention and for the foods of the invention may be included vegetable oils, marine oils such as fish oils and fish liver oils and algae. Possible vegetable oil sources include olive oil, soybean oil, canola oil, high oleic sunflower seed oil, high oleic safflower oil, safflower oil, sunflower seed oil, flaxseed (linseed) oil, corn oil, cottonseed oil, peanut oil, evening primrose oil, borage oil, and blackcurrant oil. In addition, other oils/fats, and extracts which may be encapsulated herein are palm oil, tarragon oil, clove oil, cardamom oil, thymol, carvacrol, anise, cinnamon oil, oregano oil, arnica, basil, bergamot, calendula, caraway, chamomile, cinnamon, citrus, elder, eucalyptus, fir needle, garlic, hops, juniper, lavender, lemon balm, licorice, marjoram, passionflower, peppermint, primrose, and thyme.

If desired, polyunsaturated fatty acids, and most especially omega-3 and/or omega 6 acids, may be combined with a carrier and spray dried to form a powder prior to encapsulation. An emulsion may be formed with the carrier and the unsaturated fatty acids prior to spray drying. Examples of suitable carriers include modified food starches, maltodextrins, proteins such as soy protein and caseinate, sugars and mixtures thereof. Then, the spray dried powder is encapsulated in accordance with the invention. Emulsions may be formed in a homogenizer such as a high pressure homogenizer from Invensys APV of Tonawanda, N.Y. The emulsion will typically comprise from 5 wt % to 25 wt % of carrier and 35 to 15 wt % of the unsaturated fatty acid. The emulsion typically will have about 40% solids and the balance water.

The food of the invention may be any of several foods are preferably those which could be supplemented plant sterol compounds and one or more of ingredients a-k, optionally with pro-oxidant minerals and polyunsaturated fatty acids. Preferably the food is a nutrition bar, a ready-to-drink beverage, a sweet powder such as a powdered beverage, a soup, or a frozen confection. The food components mentioned in this application may be included in the encapsulate or outside the encapsulate.

The food of the invention may include protein sources. Preferred sources of protein include sources of whey protein such as whey protein isolate and whey protein concentrate, sources of rice protein such as rice flour and rice protein concentrate, and sources of pea protein. Soy protein may also be used. The protein may be present in the food in discrete nuggets, in the encapsulate, other forms, and any combination thereof.

Additional dairy protein sources include one or more of dairy protein source, such as whole milk, skim milk, buttermilk, condensed milk, evaporated milk, milk solids non-fat, etc. The dairy source may contribute dairy fat and/or non-fat milk solids such as lactose and milk proteins, e.g. the whey proteins and caseins. Especially preferred, to minimize the caloric impact, is the addition of protein as such rather than as one component of a food ingredient such as whole milk. Preferred in this respect are protein concentrates such as one or more of whey protein concentrate as mentioned above, milk protein concentrate, caseinates such as sodium and/or calcium caseinate, isolated soy protein and soy protein concentrate. Total protein levels within the foods of the invention, particularly when the food takes the form of a nutrition bar, are preferably within the range of 3 wt % to 50 wt %, such as from 3 wt % to 30 wt %, especially from 3 wt % to 20%.

When protein nuggets are employed, they typically include greater than 50 wt % of protein selected from the group consisting of milk protein, rice protein and pea protein and mixtures thereof, especially between 51 wt % and 99 wt %, more preferably between 52 wt % and 95 wt %, most preferably 55 wt % or above. Other ingredients which may be present in the nuggets would include one or more of other proteins, such as soy protein, include lipids, especially triglyceride fats, and carbohydrates, especially starches. Particularly where the nuggets are made using the moderated temperature extrusion process described below, it is advisable that the remaining ingredients be no more sensitive to heat degradation (e.g., have the same or lower degradation point) than the selected non-soy protein.

The food of the invention may include various oils or fats whether as unsaturated fatty acids moieties encapsulated herein or elsewhere in the food. In addition to those mentioned above, such oils and fats include other vegetable fat, such as for example, cocoa butter, illipe, shea, palm kernel, cottonseed, coconut, rapeseed, and corn oils, or mixtures thereof. A blend of oils (e.g., canola, soybean, or high oleic oils) may be used, especially containing either synthetic antioxidants such as BHT, TBHQ or natural antioxidants such as mixed tocopherols, ascorbic acid and rosemary extract or a blend of the above. When the source is for linoleic and linolenic acids (C18:2 and C18:3), straight oil or blends of oil such as canola plus soybean with an appropriate antioxidant system can be used. Natural antioxidants which may be suitable include extracts from laurel and basil prepared by hydrodistillation. However, animal fats such as butter fat may also be used if consistent with the desired nutritional profile of the product.

An especially preferred blend of oils for use in the bars, pastas, powdered beverages, soups and other foods of the invention is a blend of canola and soybean oils at a weight ratio canola to soybean of from 35:65 to 65:35, especially about 50:50. This may be used within the shell or outside of the shell. The blend may be used in the bars and other foods of the invention at levels of from 2 to 25 wt %, especially from 5 to 20 wt %, most especially from 8 to 12wt %. The blend provides a good, stable source of omega-3 and omega-6 fatty acids. For instance, levels of 0.15 to 0.2 g/serving of omega-3 and 1 to 2 g per serving of omega-6 are readily provided by the canola/soybean blend in food having an excellent shelf life as long as 12 or even 14 months. The canola/soybean blend preferably includes antioxidants, in particular BHT or TBHQ or a combination of ascorbic acid and rosemary extract, preferably at levels of 50 to 3000 ppm.

In general, where encapsulated oils containing PUFA moieties are used in accordance with the invention, added antioxidants such as tocopherols, ascorbic acid and/or rosemary extract may be omitted; that is, the oils may be essentially free of added antioxidants, especially free of added antioxidants. Where non-encapsulated oils containing PUFA moieties are used, it is preferred that added antioxidants such as tocopherols, ascorbic acid and/or rosemary extract be present in the oil.

Polyunsaturated fats, particularly those containing omega-3 and omega-6 fatty acids, are preferably incorporated as encapsulates in accordance with the invention. Or, they can be incorporated into the product as oils, or in other forms such as alternative capsules or microcapsules, for example in the microcapsules of EP 648 076, the disclosure of which is incorporated by reference herein. The term “capsules” herein shall encompass encapsulates formed in accordance with the process of the invention and other encapsulating processes as well as shells into which a product has been placed. “Microcapsules” herein refers to capsules of very small size such as those of EP 648 076 and is encompassed within the term “capsules.”

In the case of a nutrition bar, preferably the amount of fat is not more than 45 wt %, especially not more than 35 wt %, preferably from 0.5 to 10 wt %, still preferably from 0.5 to 5 wt %. Carbohydrates can be used in the food of the invention at levels of from 0 to 90%, especially from 1% to 49%. In addition to sweeteners, the fibers and the carbohydrate bulking agents mentioned below, examples of suitable carbohydrates include starches such as are contained in rice flour, flour, peanut flour, tapioca flour, tapioca starch, and whole wheat flour and mixtures thereof. Where the food takes the form of a nutrition bar, levels of carbohydrates in the bar as a whole will typically comprise from 5 wt % to 90 wt %, especially from 20% to 65 wt %.

If it is desired to include a bulking agent in the food, within or external to the nuggets or capsules/microcapsules, a preferred bulking agent is inert polydextrose. Polydextrose may be obtained from Danisco under the brand name Litesse®. Other conventional bulking agents which may be used alone or in combination include maltodextrin, sugar alcohols, corn syrup solids, sugars or starches. Total bulking agent levels in the foods, e.g., nutritional bars, of the invention, will preferably be from about 0% to 20 wt %, preferably 5% to 16%.

Flavorings are preferably added to the food or nutrition bar in amounts that will impart a mild, pleasant flavor. The flavoring may be in nuggets or the encapsulates/microencapsulates or external to the nuggets and the encapsulates/microencapsulates in the bar or other food, provided that processing is not adversely affected. The flavoring may be any of the commercial flavors employed in nutrition bars or other foods, such as varying types of cocoa, pure vanilla or artificial flavor, such as vanillin, ethyl vanillin, chocolate, malt, mint, yogurt powder, extracts, spices, such as cinnamon, nutmeg and ginger, mixtures thereof, and the like. It will be appreciated that many flavor variations may be obtained by combinations of the basic flavors.

The nutrition bars or other foods are flavored to taste. Suitable flavorants may also include seasoning, such as salt (sodium chloride) or potassium chloride, and imitation fruit or chocolate flavors either singly or in any suitable combination. Flavorings which mask off-tastes from vitamins and/or minerals and other ingredients are preferably included in the products of the invention, in the encapsulates/microencapsulates, in protein nuggets and/or elsewhere in the product. Preferably, flavorants are present at from 0.25 to 3 wt % of the food, excluding salt or potassium chloride, which is generally present at from 0 to 1%, especially 0.1 to 0.5%.

The capsules, any nuggets and the bar or other food may include colorants, if desired, such as caramel colorant. Colorants are generally in the food at from 0 to 2 wt %, especially from 0.1 to 1%. Preferably the encapsulates and their contents do not contain HLB emulsifiers. Rather, any emulsifiers present in the encapsulates and their contents are emulsifying proteins or carbohydrates. The products may, however, include eggs as desired

If desired, the food, especially any nuggets, may include processing aids such as calcium chloride.

In general, it is preferred not to use emulsifiers, particularly HLB emulsifiers, in the process of making the sol-gel, but they may be present in the contents of the shell or in the overall end product e.g., nutrition bar. One example would be the emulsifiers for the oil-in-water emulsions which may be contained within the shells as described above. For instance, unsaturated oils may be emulsified with a carrier prior to spray drying. Typical emulsifying agents may be phospholipids and proteins or esters of long chain fatty acids and a polyhydric alcohol. Lecithin is an example. Fatty acid esters of glycerol, polyglycerol esters of fatty acids, sorbitan esters of fatty acids and polyoxyethylene and polyoxypropylene esters of fatty acids may be used but organoleptic properties, of course, must be considered. Mono- and di-glycerides may be used as well. Emulsifiers may be used in any emulsions used to spray dry the unsaturated fatty acids in amounts of about 0.03% to 0.3%, preferably 0.05% to 0.1%. The same emulsifiers may also be present in the nutrition bar or other food and/or protein nuggets, again at levels overall of about 0.03% to 0.3%, preferably 0.05% to 0.1%. Emulsifiers may be used in combination, as appropriate. Any nuggets may also include emulsifiers.

Among fiber sources which may be included in the foods of the invention are fructose oligosaccharides (fos) such as inulin, guar gum, gum arabic, gum acacia, oat fiber, cellulose, whole grains, and mixtures thereof. The compositions preferably contain at least 2 grams of fiber per 56 g serving, especially at least 5 grams of fiber per serving. Preferably, fiber sources are present in the product at greater than 0.5 wt. % and do not exceed 6 wt. %, especially 5 wt. %. As indicated above, additional bulking agents such as maltodextrin, sugar alcohols, corn syrup solids, sugars, starches and mixtures thereof may also be used. Total bulking agent levels in the products of the invention, including fibers and other bulking agents, but excluding sweeteners will preferably be from about 0% to 20%, especially from 1 to 15 wt %. The fiber and the bulking agent may be present in the food as a whole, e.g., the nutrition bar, and/or in capsules, nuggets, etc. provided that processing is not impaired.

Carrageenan may be included in the bars or other food of the invention, internal or external to the shells and nuggets, eg, as a thickening and/or stabilizing agent (0 to 2 wt % on product, especially 0.2 to 1%). Cellulose gel and pectin are other thickeners which may be used alone or in combination, e.g., at 0 to 10 wt %, especially from 0.5 to 2 wt %.

Typically, if the food is a nutrition bar, or in any of a number product forms which are generally sweet, the food will be naturally sweetened. The sweetener may be included in the encapsulates/microencapsulates and/or in any nuggets or elsewhere in the bar or food provided that it does not interfere with the processing of the capsule or nugget. Sources of sweetness include sucrose (liquid or solids), glucose, fructose, and corn syrup (liquid or solids), including high fructose corn syrup, corn syrup, tagatose, maltitol, corn syrup, high maltose corn syrup and mixtures thereof. Other sweeteners include leucrose, trihalose, lactose, maltose, isomaltulose, glycerine, brown sugar, sucromalt, and galactose and mixtures thereof. Polyol sweeteners other than sugars include the sugar alcohols such as maltitol, xylitol, isomalt and erythritol. Levels of sweeteners and sugar sources preferably result in sugar and/or other polyol solids levels of up to 20 wt %, especially from 10 to 17 wt % of a nutrition bar or ready to drink beverage.

Other polysaccharides which may be included in the encapsulates or elsewhere in the food include modified or unmodified starches, including dextrins, maltodextrins, and cyclodextrins and the high fiber dextrin product sold by Roquette under the name Nutriose®.

If it is desired to use artificial sweeteners, these may likewise be present in the microcapsule and/or nugget and/or within the bar or other food external to the nugget, provided that it does not interfere with processing. Any of the artificial sweeteners well known in the art may be used, such as aspartame, saccharine, Alitame® (obtainable from Pfizer), Acesulfame K (obtainable from Hoechst), cyclamates, neotame, sucralose, mixtures thereof and the like. The artificial sweeteners are used in varying amounts of about 0:005% to lwt % on the bar or other food of the invention, preferably 0.007% to. 0.73% depending on the sweetener, for example.

Aspartame may be used at a level of 0.05% to 0.15%, preferably at a level of 0.07% to 0.11%. Acesulfame K is preferred at a level of 0.09% to 0.15%.

Ingredients which, if present, will generally be found within a bar but external to the encapsulates and/or any nuggets include, but are not limited to, rolled oats, chocolate or compound chips or other chocolate or compound pieces, cookie and/or cookie dough pieces, such as oatmeal cookie pieces, brownie pieces, fruit pieces, such as dried cranberry, apple, etc., fruit jelly, vegetable pieces such as rice, honey and acidulants such as malic and citric acids, leavening agents such as sodium bicarbonate and peanut butter.

The foods of the invention may be made by known methods. The encapsulates are added to the foods at a convenient time in the processing, provided that they are not exposed to temperatures which cause degradation of their ingredients. Likewise, if protein-containing nuggets are present, the processor must be sensitive to any conditions which could cause degradation of the nugget.

Extruded nutritional bars may be made by cooking a syrup containing liquid (at ambient temperature) ingredients and then mixing with dry ingredients. The mixture is then extruded onto a conveyor belt and cut with a cutter. Any nuggets, e.g., protein nuggets, are included among the dry ingredients. The encapsulates/microencapsulates and any nuggets should only be added to the syrup when the syrup is at a temperature below that at which any of the encapsulates/microencapsulates or nugget components degrade. Syrup ingredients may include components such as corn syrup, glycerine (0-20 wt % on total product, especially 0.5 to 10 wt %), lecithin and soybean oil or other liquid oils. In addition to the capsules and any nuggets, other dry components include grains, flours (e.g., rice or peanut), maltodextrin and milk powders.

Nutritional bars in the form of granola bars may be made by cooking the syrup, adding the dry ingredients, blending the syrup and dry ingredients in a blender, feeding the blended mix through rollers and cutting with a cutter.

The bars of the invention may be coated, e.g., with milk chocolate or yogurt flavored coating. Chocolates with little or no milk or milk products may be considered so as to maximize the presence of chocolate antioxidants and, if and to the extent desired, to try to avoid reported neutralization of antioxidants in the chocolate by milk or its components.

Typically, excluding moisture lost during processing, the uncoated bars of the invention will be made from 30-50 wt % syrup, especially 35-45%, and 50-70 wt % dry ingredients, especially 55-65 wt %. Generally, coated bars according to the invention will be made from 30-50 wt % syrup, especially 35-45 wt %, 40-50 wt % dry ingredients, especially 40-45% and 0-30 wt % coating (e.g., chocolate or compound coating), especially 5-25 wt %, particularly 10-20 wt % coating.

Any nuggets preferably contain greater than 50 wt %, especially greater than 60%, more preferably greater than 70 or 80% proteins, especially non-soy proteins selected from the group consisting of milk protein, rice protein and pea protein.

It can be expected that the benefits of the invention will be realized in various types of foods, including various types of nutrition bars including, without limitation, snack bars and meal replacement bars. One example would be granola bars. Other applicable foods include soups and sweet powders which may be used to sweeten, flavor and fortify beverages such as milk, and ready-to-drink beverages.

Soups according to the invention are prepared by dry mixing the ingredients, as is known in the art. All seasoning is added to a ribbon blender (powder mixer). Mixing takes between 12 and 15 minutes depending upon the number of ingredients and size of the batch in the mixer. The mix is placed into a large tote that is taken to the packaging line.

In the case of powdered beverages, the product will typically be made using the following process. The ingredients are scaled to the quantity dictated in the formulation. The scaled ingredients are placed in a sifter placed over a 20 mesh standard screening unit. The ingredients are then bumped though the standard screen. The screened ingredients are emptied into a container, the lid is sealed and then the container is shaken vigorously for at least two minutes. The contents of the container are emptied into a 20 mesh standard screen and then stored in an air tight container. Beverages are typically prepared by scaling out the appropriate serving size of powder, scaling out 8 oz. of refrigerated skim milk, pouring milk into a blender vessel, turning the blender to a low setting and adding powder to the agitating skim milk, covering the blender vessel with an appropriate closure, increasing the speed to mid-high power, agitating at mid-high power for 20-30 seconds and then stopping agitation. The beverage is typically served and consumed shortly after preparation.

Sieve Analysis and the Saturn DigiSizer® (a Laser Light Scattering instrument using the Mie and Fraunhofer Theories with Dry Sample Dispersion) techniques may be used to measure the particle size (average particle diameter).

An example of preparation of a ready to drink beverage is as follows. Water at ambient conditions and cellulose are mixed in a high shear mixer for 5 minutes. The mix is transferred into a cold kettle A set at 50° F. The oil in water emulsion is then added. Separately, the lecithin, gums, mono & di glycerides, and water at 155° F. are high-shear-mixed for 15 minutes. Then the soy protein, calcium caseinate, Non Fat Dry Milk, and cocoa are added and mixed for 4 minutes. The mix is then transferred to a hot kettle set at 170° F. and homogenized at 500/2000 psi. Subsequently, this mix is transferred to the cold kettle A. Flavors, sugars, premix, and phosphates are dissolved in water under agitation for 5 minutes. Then the solution is sent to the cold kettle A and mixed for 15 minutes. pH is adjusted to 6.8-7.0. The solids content is then adjusted after adding the solgel. The product is then sterilized and cooled thru UHT@287° F. for 9 seconds, then cooled to 170° F. and finally homogenized 0/500 psi and filled at 70° f. in Dole cans.

DSC and TGA can be used to determine if there is a glassy state and whether there is a side reaction. Typically, a side reaction would generate a shift in the glass transition and generation of volatiles during heating. DSC may be performed on a Perkin Elmer Pyris system. The cycle consists of heating the sample from room temperature to 80° C., holding then cooling to −60° C., holding and reheating to 80° C. TGA may be performed using a Perkin Elmer TGA7. The cycle consists of heating a sample rapidly from room temperature to 140° C., then holding the sample at this temperature.

### EXAMPLE 1(a)

### Encapsulation of Canola Oil

| Ingredient | Weight [g] |
|---|---|
| Canola oil | 100 |
| Tetraethoxysilane | 250 |
| HCl (pH = 0.1) | 20 |
| NaOH (pH = 11) | 6 |

Canola oil was mixed with the TEOS (Dynasylan A™ from Degussa). The mixture was at 64° F., mixed at 10,000 rpm for three minutes. Then the hydrochloric acid was added until the temperature rose to 112° F. The sodium hydroxide solution was then slowly added. The temperature started rising and when it reached 120° F. the speed was rapidly reduced to 1000 rpm while temperature kept rising to 148° F. The exothermic reaction lasted about 20 seconds. During the reaction time ethanol was collected in a trap cooled with a mixture of dry ice and isopropyl alcohol (e.g., ˜15° F.). The speed was reduced to about 400 rpm. When the product cooled off, a solution of Ethanol 95% was added to wash off some of the sodium chloride formed.

The precipitated product was then placed in a vacuum cell at room temperature with a cold trap (˜15° F.) that retained the ethanol and volatiles. The dried product (canola oil completely encapsulated by silica) was collected 24 hours later with a 99.98% recovery of solids.

### EXAMPLE 1(b)

### Encapsulation of Phytosterol Ester and Lycopene (Prophetic)

| Ingredient | Weight [g] |
|---|---|
| Lycopene | 50 |
| Sitosterol esterified with | 50 |
| sunflower oil fatty acids | |
| Tetraethoxysilane | 250 |
| HCl (pH = 0.1) | 20 |
| NaOH (pH = 11) | 6 |

Sitosterol esters are melted. Lycopene is melted. The melts are mixed together and then mixed with the TEOS (Dynasylan A™ from Degussa) and permitted to cool. The mixture is mixed at 10,000 rpm for three minutes. Then the hydrochloric acid is added until the temperature rises to 112° F. The sodium hydroxide solution is then slowly added. The temperature starts rising and when it reaches 120° F. the speed is rapidly reduced to 1000 rpm while temperature keeps rising to 148° F. The exothermic reaction lasts about 20 seconds. During the reaction time ethanol is collected in a trap cooled with a mixture of dry ice and isopropyl alcohol (e.g., ˜15° F.). The speed is reduced to about 400 rpm. When the product cools off, a solution of Ethanol 95% is added to wash off some of the sodium chloride formed.

The precipitated product is then placed in a vacuum cell at room temperature with a cold trap (˜15° F.) that retains the ethanol and volatiles. The dried product (phytosterol esters plus lycopene completely encapsulated by silica) is collected 24 hours later.

### EXAMPLE 1(c)

### Encapsulation of Phytosterol Ester and CLA (Prophetic)

| Ingredient | Weight [g] |
|---|---|
| CLA | 50 |
| Sitosterol esterified with | 50 |
| sunflower oil fatty acids | |
| Tetraethoxysilane | 250 |
| HCl (pH = 0.1) | 20 |
| NaOH (pH = 11) | 6 |

Sitosterol esters are melted and is mixed with conjugated linoleic acid obtained from Loders Croklaan as Clarinol. The mix is then mixed with the TEOS (Dynasylan A™ from Degussa) and permitted to cool. The mixture is mixed at 10,000 rpm for three minutes. Then the hydrochloric acid is added until the temperature rises to 112° F. The sodium hydroxide solution is then slowly added. The temperature starts rising and when it reaches 120° F. the speed is rapidly reduced to 1000 rpm while temperature keeps rising to 148° F. The exothermic reaction lasts about 20 seconds. During the reaction time ethanol is collected in a trap cooled with a mixture of dry ice and isopropyl alcohol (e.g., ˜15° F.). The speed is reduced to about 400 rpm. When the product cools off, a solution of Ethanol 95% is added to wash off some of the sodium chloride formed.

The precipitated product is then placed in a vacuum cell at room temperature with a cold trap (˜15° F.) that retains the ethanol and volatiles. The dried product (phytosterol esters plus CLA completely encapsulated by silica) is collected 24 hours later.

### EXAMPLE 2(a)

The “center” of a coated bar is formed from the following components:

| Component | Wt % of Center |
|---|---|
| Protein | 25 |
| Sugar | 8 |
| Rice cereal | 16 |
| Soy protein | 6 |
| Vitamin/mineral | 4 |
| mix (including | |
| microencapsulated | |
| cupric gluconateex | |
| Wright Group) | |
| Sodium chloride | 0.5 |
| Corn syrup | 28.5 |
| Molasses | 4 |
| Peanut butter | 4 |
| Encapsulated product | 4 |
| (made by procedure | |
| of Example 1(a)) | |

The liquid components are mixed, after which the dry ingredients are added and mixed until the product is substantially homogeneous. The encapsulated product of Example 1(a) is added with the dry components. The mixture is then fed into a die and extruded at room temperature and atmospheric pressure. Upon extrusion, the bar is cut into individual serving sizes which are then coated with a chocolate confectioner's compound coating. The bar is packaged and kept at 85° F. for 12 weeks, after which it is opened and eaten. No off taste is detected. Each week of successful storage at 85° F. is believed to equate to one month of successful storage at ambient temperature.

### EXAMPLE 2(b)

### Prophetic

The “center” of a coated bar is formed from the following components:

| Component | Wt % of Center |
|---|---|
| Protein | 25 |
| Sugar | 8 |
| Rice cereal | 16 |
| Soy protein | 6 |
| Vitamin/mineral | 4 |
| mix (including | |
| microencapsulated | |
| cupric gluconateex | |
| Wright Group) | |
| Sodium chloride | 0.5 |
| Corn syrup | 28.5 |
| Molasses | 4 |
| Peanut butter | 4 |
| Encapsulated product | 4 |
| (made by procedure | |
| of Example 1(b)) | |

The liquid components are mixed, after which the dry ingredients are added and mixed until the product is substantially homogeneous. The encapsulated product of Example 1(b) is added with the dry components. The mixture is then fed into a die and extruded at room temperature and atmospheric pressure. Upon extrusion, the bar is cut into individual serving sizes which are then coated with a chocolate confectioner's compound coating. The bar is packaged and kept at 85° F. for 12 weeks, after which it is opened and eaten. No off taste is detected. Each week of successful storage at 85° F. is believed to equate to one month of successful storage at ambient temperature.

### EXAMPLE 3(a)

### Encapsulation of a 50:50 Blend of Palm Oil and Sunflower Oil

| Ingredient | Weight [g] |
|---|---|
| Palm oil | 133.08 |
| Sunflower Oil | 133.08 |
| Tetraethoxysilane | 1032.42 |
| HCl (pH = 0.1) | 10.66 |
| NaOH (pH = 11) | 28.81 |

The palm oil and the sunflower oil were blended at 95° F. (the oils needed to be heated because palm oil is solid at room temperature) and then mixed with the TEOS (Dynasylan A™ from Degussa) at 10,000 rpm for three minutes. The hydrochloric was added until the temperature rose to 110° F. The sodium hydroxide solution was then slowly added as pH in the water trap dropped from 7 to 1.8. The temperature started rising, and when it reached 122° F. the speed was rapidly reduced to 1000 rpm while temperature kept rising to 148° F. The exothermic reaction lasted about 35 seconds. During the reaction time, ethanol was collected in a trap cooled with a mixture of dry ice and isopropyl alcohol (e.g., ˜15° F.). The speed was reduced to about 400 rpm. When the product cooled off, a solution of Ethanol 95% was added to wash off some of the sodium chloride formed.

The precipitated product was then placed in a vacuum cell at room temperature with a cold trap (˜15° F.) that retained the ethanol and volatiles. The dried product was collected 24 hours later with a 99.98% recovery of solids.

### Example 3(b)

### Encapsulation of a 50:50 Blend of Palm Oil and Sunflower Oil

| Ingredient | Weight [g] |
|---|---|
| Palm oil | 183.46 |
| Sunflower oil | 185.96 |
| OSAN starch | 5.09 |
| Isomaltulose | 1.61 |
| H₂O | 9.79 |
| Tetraethoxysilane | 430.31 |
| HCl (pH = 0.1) | 82.14 |
| NaOH (pH = 11) | 104.28 |

The thick aqueous phase A containing Isomaltulose, OSAN starch, and water was made by mixing the ingredients at 4000 rpm at about 95° F. OSAN starch is starch reacted with octenylsuccinic anhydride. The oil phase B containing palm oil and sunflower oil was blended at 95° F. The aqueous solution A and the oil phase B were mixed together at 10,000 rpm to form an emulsion C. The emulsion C was then mixed with the TEOS (Dynasylan A™ from Degussa) at 10,000 rpm for three minutes. The hydrochloric acid was added until the temperature rose to 110° F. The sodium hydroxide solution was then slowly added. The temperature started rising quickly; when it reached 122° F. the speed was rapidly reduced to 1000 rpm while temperature kept rising to 148° F. The exothermic reaction lasted about 30 seconds. During the reaction time, ethanol was collected in a trap cooled with a mixture of dry ice and isopropyl alcohol (e.g ˜15° F.). The speed was reduced to about 400 rpm because of the fast formation of large clusters with an average length of 2.5 cm. When the product cooled off, a solution of Ethanol 95% was added to wash off some of the sodium chloride formed.

The precipitated product was then put in a vacuum cell at room temperature. A cold trap (˜15° F.) was set in-line to retain the ethanol and other possible volatiles. The dried product was collected 24 hours later with a total 99.99% recovery of solids.

### EXAMPLE 4

| | |
|---|---|
| Water | 325 g |
| SolGel canola | 6.5 g |

The SolGel encapsulates prepared in a similar way as those of Example 1 were added to water and stirred at room temperature. The product solubilized and formed a very thin emulsion without any precipitation. No traces of free-oil were observed. Confocal microscopy and electron microscopy showed a very thin silica layer coating the oil.

### EXAMPLE 5

| | |
|---|---|
| Skim Milk | 325 g |
| SolGel Canola | 6.5 g |

The SolGel capsules prepared in a similar way as those of Example 1 were added to cold skim milk and stirred. The product solubilized and formed a very thin emulsion without any precipitation. No traces of free oil were observed.

### EXAMPLE 6

Lipolysis experiments were prepared with pancreatin and bile on SolGel batches.

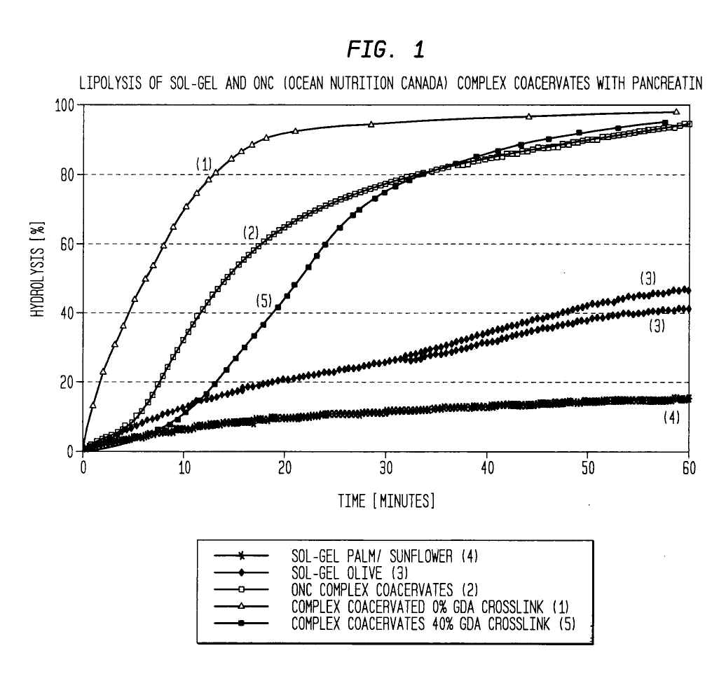

An in-vitro lypolysis test showing its efficacy to possibly deliver active ingredients in the ileum break. The results are seen in FIG. 1.

### Materials and Equipment

Bile (ox gall powder), tris(hydroxymethyl)aminomethane maleate, calcium chloride and porcine pancreatin (1× USP) were obtained from Sigma, USA.

0.10 M sodium hydroxide solution (Titrisol) was obtained from Merck, Germany.

pH stat analysis was performed on a DL55 titrator (Mettler Toledo, Switzerland) which was connected to a personal computer. Instrument control was performed via LabX light titration software. The incubation vessel of the titrator (60 ml) was thermo stated with a M3 water batch (Lauda, Germany).

### Standard Lipolysis Assay

The measuring principle of this assay is as follows: pancreatin is a broad mixture of different types of enzymes with proteases and lipases as the predominant ones. When SolGel is added to the incubation mixture, a slow release of triglycides is observed. Secondly, the free oil droplets are hydrolysed by lipases of the pancreatin in free fatty acids and monoglycerides. The produced free fatty acids will result in a drop of the pH of the incubation solution, which is automatically compensated by the addition of sodium hydroxide solution via the titrator (pH stat mode). The molar amount of sodium hydroxide solution added is equal to the molar amount of fatty acids formed during the hydrolysis of triglycerides. For calculation of the degree of hydrolysis of triglycerides it was assumed that pancreatic lipase hydrolyses 1 mole of triglycerides into 2 moles of fatty acids and 1 mole of mono-glycerides. This is the sodium hydroxide use as the measuring signal.

For lipolysis measurements, an incubation buffer of 40 mM sodium chloride, 2 mM tris (hydroxymethyl) aminomethane maleate and 80 mM calcium chloride was prepared. The pH of the buffer was adjusted with 0.10 M sodium hydroxide solution to 6.8. For each lipolysis assay 40 ml of incubation buffer, 1.0 g of bile and 500 mg of SolGel were mixed. The lipolysis reaction was started with the addition of 25 mg of pancreatin powder. During the incubation period of 60 minutes, automatic addition of sodium hydroxide solution was performed to maintain a pH value of 6.8 (pH stat). The temperature during the lipolysis assay was kept constant at 37° C.

A graph showing the results is presented in FIG. 1.

GDA is glutardialdehyde.

The graph shows that the olive and palm/sunflower oils encapsulated via a sol gel procedure using an acid catalyst (#3 and #4) showed slower oil release than for various complex coacervates. No glassy state was observed for the solgels. The solgel fully encapsulated the oil or active ingredient. The reactions were controlled to produce solgel particles greater than 1 micron. The results suggest that the solgels could be able to reach the ileum break more efficaciously.

### EXAMPLE 7

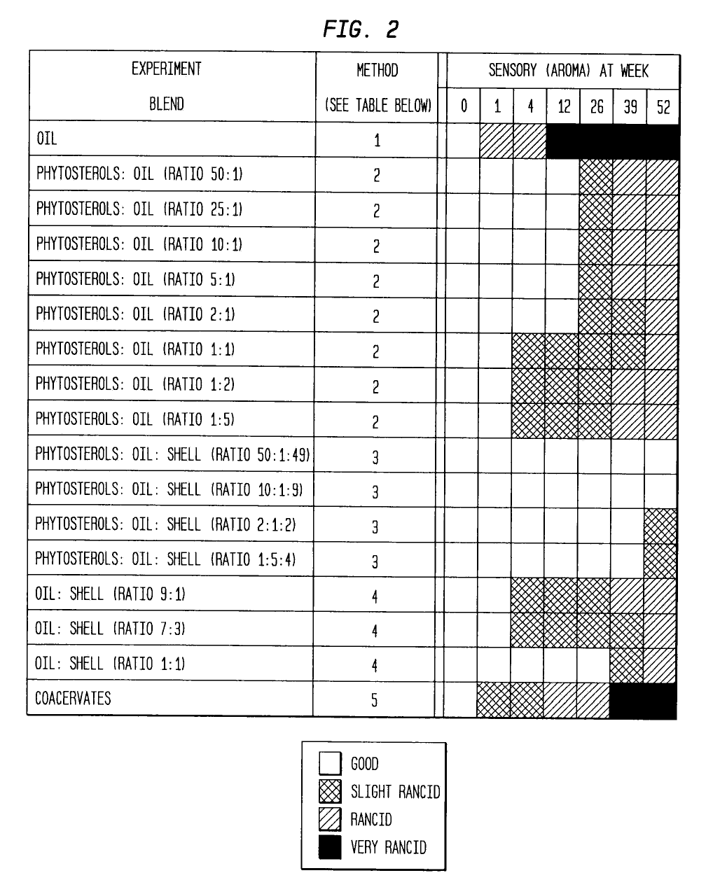

The stability of fish oil in various environments was tested as shown in FIG. 2. The greatest stability was seen where the oil was encapsulated together with phytosterols. The capsules were prepared using the sol gel method of the invention according to the method indicated in the graph (one of the following methods 1-5).

| Method | Description |
|---|---|
| 1 | 100 g of Unimeg-38 (fish oil with TDS XOU38.01/Unilever) from Ocean Nutrition of |
| | Dartmouth, Nova Scotia, Canada (ONC) were poured into 200 g glass containers. The |
| | jars were kept open (without the lid) in an incubator at 25° C. and 75% relative |
| | humidity. Sensory tests were conducted at 0, 1, 2, 4, 12, 26, 39, and 52 weeks. |
| 2 | Phytosterols were poured in a glass container and melted at about 85° C.. The fish oil |
| | from ONC was added and mixed. The blend was rapidly cooled to 15° C.. The mass was |
| | cryo-milled using liquid nitrogen. The fine powder was maintained in a dry chamber |
| | (~0% relative humidity) to avoid water condensation while being packed under |
| | vacuum. Once the system reached room temperature, 100 g samples were poured |
| | into 200 g glass containers. The jars were kept open (without the lid) in an incubator |
| | at 25° C. and 75% relative humidity. Sensory tests were conducted at 0, 1, 2, 4, 12, |
| | 26, 39, and 52 weeks. |
| 3 | SolGels were prepared using samples from method 2 above following similar |
| | procedure as shown in Example 1 above. 100 g samples were poured into 200 g glass |
| | containers. The jars were kept open (without the lid) in an incubator at 25° C. and 75% |
| | relative humidity. Sensory tests were conducted at 0, 1, 2, 4, 12, 26, 39, and 52 |
| | weeks. |
| 4 | SolGels using Unimeg-38 (fish oil with TDS XOU38.01/Unilever) from Ocean Nutrition |
| | Canada (ONC) were prepared following a similar procedure as the one depicted on |
| | Example 1 above. 100 g samples were poured into 200 g glass containers. The jars |
| | were kept open (without the lid) in an incubator at 25° C. and 75% relative humidity. |
| | Sensory tests were conducted at 0, 1, 2, 4, 12, 26, 39, and 52 weeks. |
| 5 | 100 g of Meg-3 (Omega-3 DHA powder, TDS MC60TDHA-NGH.01) from Ocean |
| | Nutrition were poured into 200 g glass containers. The jars were kept open (without |
| | the lid) in an incubator at 25° C. and 75% a relative humidity. Sensory tests were |
| | conducted at 0, 1, 2, 4, 12, 26, 39, and 52 weeks. |

### EXAMPLE 8

A meal replacer beverage having the following composition is prepared by the process indicated below.

| Component | % wt |
|---|---|
| Water | 80 |
| Vitamin mineral mix | 0.5 |
| Mono & Di glycerides | 0.2 |
| Flavor | 0.4 |
| Phosphates | 0.28 |
| Lecithin | 0.095 |
| Gums | 0.715 |
| Non Fat Dry Milk | 6.1 |
| Cellulose | 0.6 |
| Cocoa | 1.2 |
| Sugars | 2.1 |
| Caseinate | 0.6 |
| Soy Protein | 0.018 |
| Oil in water Emulsion | 0.0-6.5 |
| SolGel | 0.5-4 |

### Process

### Mix and Blend of Batch

Water at ambient conditions and cellulose are mixed in a high shear mixer for 5 minutes. The mix is transferred into a cold kettle A set at 50°. The oil in water emulsion is then added. Separately, the lecithin, gums, mono & di glycerides, and water at 155° F. are high-shear-mixed for 15 minutes. Then the soy protein, calcium casinate, Non Fat Dry Milk, and cocoa are added and mixed for 4 minutes. The mix is then transferred to a hot kettle set at 170° F. and homogenized at 500/2000 psi. Subsequently, this mix is transferred to the cold kettle A. Flavors, sugars, premix, and phosphates are dissolved in water under agitation for 5 minutes. Then the solution is sent to the cold kettle A and mixed for 15 minutes. pH is adjusted to 6.8-7.0. The solids content is then adjusted after adding the solgel. The product is then sterilized and cooled thru UHT@287° F. for 9 seconds, then cooled to 170° F. and finally homogenized 0/500 psi and filled at 70° f. in Dole cans.

If desired, any of the components which are mentioned for encapsulation herein may also be present, alone or in combination, in the product outside of capsules in addition to or instead of in the encapsulated form. However, the benefits of encapsulation have been pointed out herein.

By HLB emulsifier is meant an emulsifier having an HLB value. Therefore, as used herein “HLB emulsifiers” excludes proteins, sterols, etc. which have an emulsifying action but which lack an HLB value.

It will be appreciated that when fatty acids are mentioned herein, generally these will be present in the form of glycerides such as mono-, di- and triglycerides. Therefore, “fatty acids” encompasses glycerides containing them. The saturated fatty acid content refers to the weight percentage of saturated fatty acid residues in the oil. The term “unsaturated fatty acid content” refers to the weight percentage of unsaturated fatty acid residues in the oil. The term “monounsaturated fatty acid content” means the weight percentage of monounsaturated fatty acid residues in the oil. “Polyunsaturated fatty acid content” refers to the weight percentage of polyunsaturated fatty acid residues in the oil.

All ranges stated herein include all individual values and subranges of said values.

Unless stated otherwise or required by context, the terms “fat” and “oil” are used interchangeably herein. Unless otherwise stated or required by context, percentages are by weight.

As used herein “essentially free” and “substantial absence” mean that less than 0.1 wt % of that ingredient is present.

The word “comprising” is used herein as “including, but not limited to” the specified ingredients. The words “including” and “having” are used synonymously.

It should be understood of course that the specific forms of the invention herein illustrated and described are intended to be representative only, as certain changes may be made therein without departing from the clear teaching of the disclosure. Accordingly, reference should be made to the appended claims in determining the full scope.

What is claimed is:

1. An encapsulate comprising an outer shell and an inner core, the inner core comprising an ingredient selected from plant sterol compounds and at least one of the following groups: a-g:
a) One or more catechins;
b) A rhubarb extract
c) quinine
d) a pharmaceutical
e) minerals
f) Astaxanthine, Zeaxanthin and/or lutein
g) CLA.

2. The encapsulate according to claim 1 wherein the outer shell comprises polymerized monomers of metal- or semi-metal alkoxide.

3. The encapsulate according to claim 2 wherein the alkoxide is an alkoxide of zirconium, silicon, titanium, aluminum and/or boron.

4. The encapsulate according to claim 3 wherein at least one silicon-based alkoxide is used to produce a silicon-based polymer.

5. The encapsulate according to claim 1 wherein the inner core comprises CLA.

6. A food product comprising the encapsulate of claim 1.

7. A process of making a food product comprising mixing the encapsulate according to claim 1 with other food ingredients.

8. A nutrition bar comprising the encapsulate of claim 1.

9. A ready-to-drink beverage comprising the encapsulate of claim 1.

10. A sweet powder comprising the encapsulate of claim 1.

11. An encapsulate comprising an outer shell and an inner core, the inner core comprising an ingredient selected from plant sterol compounds and at least one of the following group's: a-k:
a) One or more Amino acids;
b) One or more catechins;
c) A rhubarb extract
d) quinine
e) a pharmaceutical
f) vitamins and/or minerals
g) A protein or proteinaceous material
h) A peptide or a polypeptide
i) Lycopene, Astaxanthine, Zeaxanthin and/or lutein
j) CLA,
k) Polyunsaturated fatty acids, said encapusulate being made using sol gel techniques.

12. The encapsulate according to claim 11 wherein the outer shell comprises polymerized monomers of metal- or semi-metal alkoxide.

13. The encapsulate according to claim 12 wherein the alkoxide is an alkoxide of zirconium, silicon, titanium, aluminum and/or boron.

14. The encapsulate according to claim 13 wherein at least one silicon-based alkoxide is used to produce a silicon-based polymer.

15. The encapsulate according to claim 11 comprising an oil in water emulsion comprising 15-70 wt % of an oil having a saturated fatty acid content of at least 20 wt % and at least 10 wt % of the oil being solid at 37° C., 0.1-5 wt % protein emulsifier; and 25-85 wt % water.

16. A food product comprising the encapsulate of claim 11.

17. A process of making a food product comprising mixing the encapsulate according to claim 11 with other food ingredients.

18. A nutrition bar comprising the encapsulate of claim 11.

19. A ready-to-drink beverage comprising the encapsulate of claim 11.

20. A sweet powder comprising the encapsulate of claim 11.

21. The encapsulate according to claim 11 wherein the polyunsaturated fatty acids comprise one or more omega-3 fatty acids.
